SEQUENCE LISTING (6 sequences)

SEQ ID NO: 1 - DNA, 25 bp, artificial sequence
  sgRNA SUV39H1 F5′
caccgccacctggggcggatcaccg

SEQ ID NO: 2 - DNA, 25 bp, artificial
  sgRNA SUV39H1 F3′
aaaccggtgatccgccccaggtggc

SEQ ID NO: 3 - DNA, 25 bp, artificial
  sgRNA SETDB1 F5′
caccgccatagcttcacgaagctgt

SEQ ID NO: 4 - DNA, 25 bp, artificial
  sgRNA SETDB1 F3′
aaacacagcttcgtgaagctatggc

SEQ ID NO: 5 - DNA, 25 bp, artificial
  sgRNA Sting F5′
caccgagcggtgacctctgggccgt

SEQ ID NO: 6 - DNA, 25 bp, artificial
  sgRNA Sting F3′
aaacacggcccagaggtcaccgctc

(19) United States
(12) Patent Application Publication
(10) Pub. No.: US 2020/0405853 A1
(43) Pub. Date: Dec. 31, 2020

(54) INHIBITOR OF SETDB1 HISTONE METHYLTRANSFERASE FOR USE IN CANCER COMBINATION THERAPY

(71) Applicants: INSTITUT CURIE, Paris (FR); INSERM, Paris (FR)

(72) Inventors: Sebastian AMIGORENA, Paris (FR); Marianne BURBAGE, Paris (FR); Derek ROOKHUIZEN, Paris (FR)

(21) Appl. No.: 16/978,285

(22) PCT Filed: Mar. 6, 2019
(86) PCT No.: PCT/EP2019/055536

§ 371 Date: Sep. 4, 2020

(30) Foreign Application Priority Data
Mar. 6, 2018 (EP) 18305234.9

(51) Int. Cl. A61K 39/395; A61K 31/713; A61P 35/00

(52) U.S. Cl. CPC A61K 39/3955; A61K 2039/505; A61P 35/00; A61K 31/713

(57) ABSTRACT

The present invention relates to an inhibitor of H3K9 histone methyl transferase SETDB1 for use in combination with at least one immune checkpoint modulator in the treatment of cancer.

### FIELD OF THE INVENTION

The present invention relates to the treatment of cancer and in particular to the use of an inhibitor of SETDB1 in combination with immune checkpoint therapy.

### BACKGROUND OF THE INVENTION

Immune checkpoints refer to a plethora of inhibitory and stimulatory pathways hardwired into the immune system that are crucial for maintaining self-tolerance and modulating the duration and amplitude of physiological immune responses in peripheral tissues, in order to minimize collateral tissue damage. Indeed, the balance between inhibitory and stimulatory signals determines the lymphocyte activation and consequently regulates the immune response (Pardoll D M, Nat Rev Cancer. 2012 Mar. 22; 12(4):252-64).

It is now clear that tumors co-opt certain immune-checkpoint pathways as a major mechanism of immune resistance, particularly against T cells that are specific for tumor antigens. Because many of the immune checkpoints are initiated by ligand-receptor interactions, they can be readily blocked by antibodies or modulated by recombinant forms of ligands or receptors. Thus agonists of co-stimulatory receptors or antagonists of inhibitory signals, both of which result in the amplification of antigen-specific T cell responses are the primary agent in current clinical testing.

In this context, cancer immunotherapy has been viewed as breakthrough in the field of cancer treatment, switching from targeting the tumor to targeting the immune system (Couzin-Frankel J., Science. 2013 Dec. 20; 342(6165):1432-3). The blockade of immune checkpoints with antibodies anti-CTLA-4, PD1 and PD-L1 has given promising clinical results and manageable safety profiles.

However, only a small proportion of patients respond to these therapies, thus, there is a need to improve cancer immunotherapies by new approaches and/or by combining anti-checkpoint antibodies with other treatments (see notably Jenkins R W et al., BJC 2018 118, 9-16 and Sharma P et al., Cell 2017; 168(4):707-723). Moreover, anti-checkpoint antibodies can induce side effects, mainly autoimmunity, such that implementing combination therapies which may help lower the administered doses, and consequently the adverse events, remains of invaluable medical help.

“Epigenetics” is defined as heritable alterations in gene expression arising from chemical changes in DNA or histone proteins. Epigenetic events include DNA methylation, covalent histone modifications and non-covalent mechanisms like integration of histone variants, nucleosome positioning and remodeling.

Methylation of histone lysine and arginine residues is regulated by two classes of enzymes with opposing activities: histone methyltransferases and histone demethylases.

Histone methyltransferases (HMT) are histone-modifying enzymes (e.g., histone-lysine N-methyltransferases and histone-arginine N-methyltransferases), that catalyze the transfer of one, two, or three methyl groups to lysine and arginine residues of histone proteins. The attachment of methyl groups occurs predominantly at specific lysine or arginine residues on histones H3 and H4. The class of lysine-specific histone methyltransferases is further subdivided into SET domain-containing and non-SET domain-containing. Methylation of the N-terminal lysine residues of histone H3, notably in position 4, 9, 27, 36 and 79 to form mono-, di-, or tri-methylated lysines, is highly documented. More than 30 histone methyltransferases have currently been described.

Epigenetic factors have been implicated in cancer, inflammatory and autoimmune diseases, and in the past few years have been recognized as promising targets for drug development. The activity of several histone methyltransferases that methylate various lysine residues of histone H3 or H4 have been associated with cancers, such as MLL, SMYDD3, G9a, Suv39H1, STDB1, EZH2, NSD3, DNS1, DOT1L, SET8, SUV420H1, SUV420H2. Conversely, various demethylases have also been involved in cancers (Morera L et al., Targeting histone methyltransferases and demethylases in clinical trials for cancer therapy, Clinical Epigenetics 2016; 8:57). Inhibitors of the histone methyltransferase, EZH2, have been proposed for the treatment of patients with relapsed or refractory B-cell lymphoma (Nature. 2012 Dec. 6; 492(7427):108-12). Inhibitors of DNA methyltransferase (DNMT) or of histone deacetylase (HDAC) are also currently approved for clinical use in the treatment of haematological malignancies. Two cytidine analogs, azacitidine (5-azacitidine or aza) and decitabine, non-specifically inhibit DNA methyltransferase activity upon incorporation into DNA, resulting in loss of DNA methylation. Both of these agents are approved for use in patients with myelodysplastic syndrome (MDS). Aza treatment results in reduced DNA methylation as demonstrated by several studies in vivo and in vitro, although the degree of demethylation seems to be limited (Magnus Tobiasson et al., Comprehensive mapping of the effects of azacitidine on DNA methylation, repressive/permissive histone marks and gene expression in primary cells from patients with MDS and MDS related disease Oncotarget, 2017, Vol. 8, (No. 17), pp: 28812-28825).

The use of inhibitors of DNMT or HDAC has also been recently proposed in combination with other cancer therapies such as immunotherapy (WO2015035112, Chiapinelli K B et al., Cell. 2015 Aug. 27; 162(5):974-86; Licht J D Cell. 2015 Aug. 27; 162(5):938-9, but see also Sharma P et al., Cell 2017 as previously mentioned). Indeed, it has been suggested that DNA demethylating agents may prime solid tumors to T-cell-mediated immune response and, therefore, may work in synergy with antitumor immunotherapy, such as checkpoint inhibitors (Roulois D, Yau H L, De Carvalho D D. Pharmacological DNA demethylation: Implications for cancer immunotherapy. Oncoimmunology. 2016; 5(3):e1090077). Also, incidental clinical findings suggest that non-small-cell carcinoma lung cancer patients pre-treated with 5-Azacytidine have better clinical response to subsequent anti-PD1 therapy. (Juergens R A, Wrangle J, Vendetti F P, Murphy S C, Zhao M, Coleman B, Sebree R, Rodres K, Hooker C M, Franco N et al. Combination epigenetic therapy has efficacy in patients with refractory advanced non-small cell lung cancer. Cancer Discov 2011; 1:598-607) and mice models of melanoma (B16) do respond better to the combination of 5-Azacytidine plus anti-CTLA4 than 5-Azacytidine alone or anti-CTLA4 alone (see Chiappinelli K B et al. Inhibiting DNA Methylation Causes an Interferon Response in Cancer via dsRNA Including Endogenous Retroviruses. Cell 2015; 162:974-86; and Roulois D et al. DNA-Demethylating Agents Target Colorectal Cancer Cells by Inducing Viral Mimicry by Endogenous Transcripts. Cell 2015; 162:961-973; PMID:26317465;)

However, the role of such epigenetic modulators in cancer immunology and immunotherapy remains however poorly understood. Indeed, the effects of demethylating agents are diverse, and identification of genes, whose reactivation predicts or mediates response, remains elusive. Typically, immune modulatory effects of treatment with 5-Azacytidine, a DNMT, are complex and dependent on the clinical setting and type of patients (see Frösig T M and Hadrup S R, Mediators Inflamm. 2015; 2015: 871641).

Thus there remains a need for implementing combination therapies that may improve efficacy of cancer immunotherapies with limiting adverse side effects.

### SUMMARY OF THE INVENTION

The present inventors have demonstrated for the first time that the anti-tumor effect of an immune checkpoint modulator is greatly enhanced in the absence of SETDB1. In particular, they show that surprisingly, while anti-PD1 treatment, or suppression of SETDB1 have only moderate or even lacks anti-tumor effects separately, their combination leads to a massive and sustained tumor growth inhibition. Furthermore the inventors also surprisingly suggest that combination of an immune checkpoint inhibitor, (such as an anti-PD1 or an anti PDL1) together with SETDB1 inhibition would be drastically more efficient than the combination with Suv39H1, although the later combination was already shown to synergistically improve anti-PD1 efficiency. This observation is quite surprising as both methyltransferases are known to trimethylate H3K9. As mentioned above, numerous epigenetic factors have been described and potentially involved in cancer development. The present results demonstrate that identification of synergistic combination between potential therapeutic targets cannot be expected from their known individual role in the pathophysiological cascades.

Thus the present invention relates to an inhibitor of H3K9 histone methyl transferase SETDB1 for use in combination with at least one modulator of an immune checkpoint protein in the treatment of cancer in a patient.

### Definitions

“Treatment”, or “treating” as used herein, is defined as the application or administration of a therapeutic agent or combination of therapeutic agents (e.g., an inhibitor of SETDB1 and/or an immune checkpoint modulator) to a patient, or application or administration of said therapeutic agents to an isolated tissue or cell line from a patient, who has a cancer with the purpose to cure, heal, alleviate, relieve, alter, remedy, ameliorate, improve or affect the cancer, or any symptom of the cancer. In particular, the terms “treat” or treatment” refers to reducing or alleviating at least one adverse clinical symptom associated with cancer, e.g., pain, swelling, low blood count etc.

In another embodiment, the term “treat” or treatment” refers to slowing or reversing the progression neoplastic uncontrolled cell multiplication, i.e. shrinking existing tumors and/or halting tumor growth.

The term “treat’ or treatment” also refers to inducing apoptosis in cancer or tumor cells in the subject.

The term “treatment” or “treating” is also used herein in the context of administering the therapeutic agents prophylactically.

The term “effective dose” or “effective dosage” is defined as an amount sufficient to achieve, or at least partially achieve, the desired effect. The term “therapeutically effective dose” is defined as an amount sufficient to cure or at least partially arrest the disease and its complications in a patient already suffering from the disease. The term “patient” includes human and other mammalian subjects that receive either prophylactic or therapeutic treatment.

As used herein, the term “therapeutically effective regimen” refers to a regimen for dosing, timing, frequency, and duration of the administration of one or more therapies according to the invention (i.e., the inhibitor of SETDB1 and the at least one immune checkpoint modulator), for the treatment and/or the management of cancer or a symptom thereof. In a specific embodiment, the regimen achieves one, two, three, or more of the following results: (1) a stabilization, reduction or elimination in the cancer cell population; (2) a stabilization or reduction in the growth of a tumor or neoplasm; (3) an impairment in the formation of a tumor; (4) eradication, removal, or control of primary, regional and/or metastatic cancer; (5) a reduction in mortality; (6) an increase in disease-free, relapse-free, progression-free, and/or overall survival, duration, or rate; (7) an increase in the response rate, the durability of response, or number of patients who respond or are in remission; (8) a decrease in hospitalization rate, (9) a decrease in hospitalization lengths, (10) the size of the tumor is maintained and does not increase or increases by less than 10%, preferably less than 5%, preferably less than 4%, preferably less than 2%, and (11) an increase in the number of patients in remission.

As used herein, the term “in combination”, or “combined administration” in the context of the invention refers to the administration of an inhibitor of SETDB1 and of at least one immune checkpoint modulator to a patient for cancer therapeutic benefit. The term “in combination” in the context of the administration can also refer to the prophylactic use of a SETDB1 inhibitor when used with at least one immune checkpoint modulator.

The use of the term “in combination” does not restrict the order in which the therapies (e.g., SETDB1 and the at least one immune checkpoint modulator) are administered to a subject. A therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a patient which had, has, or is susceptible to cancer. The therapies are administered to a patient in a sequence and within a time interval such that the therapies can act together. In a particular embodiment, the therapies are administered to a subject in a sequence and within a time interval such that they provide an increased benefit than if they were administered otherwise. Any additional therapy can be administered in any order with the other additional therapy.

These results of the present invention have established a basis for dual treatment of patients with an inhibitor of SETDB1 and at least one immune checkpoint modulator such as an anti-PD-1 antibody. These two therapies need not be given concurrently, but can also be given sequentially, for example beginning with the SETDB1 inhibitor and followed by immune checkpoint modulation. Accordingly, and as used herein, the expression “An inhibitor of H3K9 histone methyl transferase SETDB1 for use in combination with at least one immune checkpoint modulator in the treatment of cancer” can be used interchangeably with the expression “At least one immune checkpoint modulator for use in combination with an inhibitor of H3K9 histone methyl transferase SETDB1 in the treatment of cancer”.

The terms “synergy,” “synergistic,” or “synergistic effect” as used herein describe an effect that has a magnitude that is greater than the sum if the individual effects. In some embodiments of the present invention, the use of both a SETDB1 inhibitor and an immune checkpoint modulator in concert provides a synergistic therapeutic effect on a neoplastic condition in a patient and/or on the growth of a cell. For example, if use of a SETDB1 inhibitor produced a 10% reduction in tumor growth and use of an immune checkpoint modulator alone produced a 20% reduction in tumor growth, then the additive effect for reducing neoplastic or tumor growth would be 30% reduction. Hence, by comparison, a synergistic effect when using both the inhibitor of SETDB1 and the immune checkpoint modulator would be reduction in tumor or neoplastic growth to any extent greater than 30% reduction.

As used herein, the term “antibody” refers to a protein that includes at least one immunoglobulin variable region, e.g., an amino acid sequence that provides an immunoglobulin variable domain or an immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab fragments, F(ab′)2 fragments, Fd fragments, Fv fragments, and dAb fragments) as well as complete antibodies, e.g., intact and/or full length immunoglobulins of types IgA, IgG (e.g., IgGI, IgG2, IgG3, IgG4), IgE, IgD, IgM (as well as subtypes thereof). The light chains of the immunoglobulin may be of kappa or lambda types. In one embodiment, the antibody is glycosylated. An antibody can be functional for antibody-dependent cytotoxicity and/or complement-mediated cytotoxicity, or may be non-functional for one or both of these activities.

### DETAILED DESCRIPTION OF THE INVENTION

### Inhibitors of SETDB1

As used herein the term “SET Domain Bifurcated1” or “SETDB1” or “H3K9 histone methyl transferase SETDB1” (also known as ESET, KG1T, KIAA0067, KMT1E, TDRD21) has its usual meaning in the art and refers to a histone methyl transferase that methylates lysine in position 9 of histone H3 (H3K9) (Loyola A et al. EMBO Reports. 2009; 10(7):769-775; Gurard-Levin Z A et al., Annu Rev Biochem. 2014; 83:487-517.)

SETDB1 is a member of the SET domain-containing proteins involved in histone methylation, which are present in all eukaryotes. This protein family is characterized by a SET domain comprised of approximately 130 amino acids, which was named after the three Drosophila proteins suppressor of variegation 3-9 (Su(var)3-9), enhancer of zeste (E(z)), and homeobox gene regulator trithorax (Trx). The SET domain methylates the ε-amino group of lysine residues using the cofactor S-adenosyl-L-methionine (SAM) during this process.

The human SETDB1 gene (referenced ENSG00000143379 in the database Ensembl), mapped onto human chromosome 1q21. The human SETDB1 gene consists of three isoforms. Isoform 1 encoded by the longest transcript consists of all intact domains and is expressed ubiquitously. Isoform 2 is a shorter protein compared to isoform 1 (due to the use of an alternate in-frame splice site in the 3′coding region), while isoform 3 has a distinct short C-terminus and lacks the HMT and SET domains, as compared to isoform 1.

The SETDB1 protein including the 3 isoforms (produced by alternative splicing) is referenced under number Q15047 in UNIPROT. The protein (isoform 1 identified as the canonical sequence) consists of 1291 amino acids and possesses a molecular mass of 143.1 kDa. Human and mouse SETDB1 gene showed 92% similarity at the amino acid level and contain 22 exons. SETDB1 comprises a C-terminal region which constitutes an evolutionarily conserved SET, pre-SET, and post-SET domains involved in histone methylation. The catalytic activity of the SET domain is embedded in the pre- and the post-SET domains. The promoter region of mouse SETDB1 gene is rich in GC content and contains binding regions for GATA-binding factor 1 (GATA-1), nuclear factor Y (NF-Y) and specificity protein-1 (Sp-1) proteins that are characterized housekeeping genes.

According to the present invention, the general term SETDB1 also encompasses all orthologues of the human SETDB1 protein.

As per the invention, an inhibitor of SETDB1 can be selected among any natural compound or not having the ability to inhibit SETDB1 activity or gene expression.

The inhibiting activity of a compound may be determined using various methods as described in Greiner D. Et al. Nat Chem Biol. 2005 August; l(3): 143-5 or Eskeland, R. et al. Biochemistry 43, 3740-3749 (2004). Typically an inhibitor of SETDB1 refers to a compound that inhibits the SET DB1 activity by at least 20%, 30%, 40%, 50%, 60% and preferably more than 70%, even more preferably more than 80%, more than 90%, more than 95%, more than 99% or even 100% (corresponding to no detectable activity) in a subject (or in a cell in vitro) as compared to the SETDB1 activity prior to or in the absence or, administration of said compound.

The inhibitor of SETDB1 can be selected from small organic molecules, aptamers, intrabodies, polypeptides or inhibitors of H3K9 histone methyl transferase SETDB1 gene expression (Bennett R L, Licht J D. “Targeting Epigenetics in Cancer. Annu Rev Pharmacol Toxicol.” 2018 Jan. 6; 58:187-207; Karanth A V et al., “Emerging role of SETDB1 as a therapeutic target”. Expert Opin Ther Targets. 2017 March; 21(3):319-331.

Typically, the inhibitor of H3K9-histone methyltransferase SETDB1 is a small organic molecule. The term “small organic molecule” refers to a molecule of a size comparable to those organic molecules generally used in pharmaceuticals. The term excludes biological macro molecules (a g., proteins, nucleic acids, etc.). Preferred small organic molecules range in size up to about 5000 Da, more preferably up to 2000 Da, and most preferably up to about 1000 Da.

In a particular embodiment, the inhibitor of H3K9-histone methyltransferase SETDB1 can be mithramycin (also referred to as plicamycin, MIT) (Ryu H et al., “ESET/SETDB1 gene expression and histone H3 (K9) trimethylation in Huntington's disease”; Proc Natl Acad Sci USA. 2006 Dec. 12; 103(50):19176-81). In some embodiment mithramycin may be combined with cystamine.

Identification of new small molecule inhibitors can be achieved according to classical techniques in the field. The current prevailing approach to identify hit compounds is through the use of a high throughput screen (HTS). Small molecules agents can be identified from within a small molecule library, which can be obtained from commercial sources such as AMRI (Albany, N.Y.), AsisChem Inc. (Cambridge, Mass.), TimTec (Newark, Del.), among others, or from libraries as known in the art.

In another embodiment, the SETDB1 inhibitor is an aptamer. Aptamers are a class of molecule that represents an alternative to antibodies in term of molecular recognition.

Aptamers are oligonucleotide or oligopeptide sequences with the capacity to recognize virtually any class of target molecules with high affinity and specificity. Such ligands may be isolated through Systematic Evolution of Ligands by Exponential enrichment (SELEX) of a random sequence library, as described in Tuerk C. and Gold L., 1990. The random sequence library is obtainable by combinatorial chemical synthesis of DNA.

In this library, each member is a linear oligomer of a unique sequence that is optionally chemically modified. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas P, Cohen B, Jessen T, Grishina I, McCoy J, Brent R. “Genetic selection of peptide aptamers that recognize and inhibit cyclin-dependent kinase 2”. Nature. 1996 Apr. 11; 380(6574):548-50).

Inhibition of SETDB1 in a cell according to the invention may also be achieved with intrabodies. Intrabodies are antibodies that bind intracellularly to their antigen after being produced in the same cell (for a review see for example, Marschall A L, Dübel S and Böldicke T “Specific in vivo knockdown of protein function by intrabodies”. MAbs. 2015; 7(6):1010-35, but see also Van Impe K, Bethuyne J, Cool S, Impens F, Ruano-Gallego D, De Wever O, Vanloo B, Van Troys M, Lambein K, Boucherie C, et al. “A nanobody targeting the F-actin capping protein CapG restrains breast cancer metastasis”. Breast Cancer Res 2013; 15:R116; Hyland S, Beerli R R, Barbas C F, Hynes N E, Wels W. “Generation and functional characterization of intracellular antibodies interacting with the kinase domain of human EGF receptor. Oncogene 2003; 22:1557-67”. Lobato M N, Rabbitts T H. “Intracellular antibodies and challenges facing their use as therapeutic agents”. Trends Mol Med 2003; 9:390-6, and Donini M, Morea V, Desiderio A, Pashkoulov D, Villani M E, Tramontano A, Benvenuto E. “Engineering stable cytoplasmic intrabodies with designed specificity”. J Mol Biol. 2003 Jul. 4; 330(2):323-32.).

Intrabodies can be generated by cloning the respective cDNA from an existing hybridoma clone or more conveniently, new scFvs/Fabs can be selected from in vitro display techniques such as phage display which provide the necessary gene encoding the antibody from the onset and allow a more detailed predesign of antibody fine specificity. In addition, bacterial-, yeast-, mammalian cell surface display and ribosome display can be employed. However, the most commonly used in vitro display system for selection of specific antibodies is phage display. In a procedure called panning (affinity selection), recombinant antibody phages are selected by incubation of the antibody phage repertoire with the antigen. This process is repeated several times leading to enriched antibody repertoires comprising specific antigen binders to almost any possible target. To date, in vitro assembled recombinant human antibody libraries have already yielded thousands of novel recombinant antibody fragments. It is to be noted that the prerequisite for a specific protein knockdown by a cytoplasmic intrabody is that the antigen is neutralized/inactivated through the antibody binding. Five different approaches to generate suitable antibodies have emerged: 1) In vivo selection of functional intrabodies in eukaryotes such as yeast and in prokaryotes such as E. coli (antigen-dependent and independent); 2) generation of antibody fusion proteins for improving cytosolic stability; 3) use of special frameworks for improving cytosolic stability (e.g., by grafting CDRs or introduction of synthetic CDRs in stable antibody frameworks); 4) use of single domain antibodies for improved cytosolic stability; and 5) selection of disulfide bond free stable intrabodies. Those approaches are notably detailed in Marschall, A. L et al., mAbs 2015 as mentioned above.

The most commonly used format for intrabodies is the scFv, which consists of the H- and L-chain variable antibody domain (VH and VL) held together by a short, flexible linker sequence (frequently (Gly4Ser)3), to avoid the need for separate expression and assembly of the 2 antibody chains of a full IgG or Fab molecule. Alternatively, the Fab format comprising additionally the C1 domain of the heavy chain and the constant region of the light chain has been used. Recently, a new possible format for intrabodies, the scFab, has been described. The scFab format promises easier subcloning of available Fab genes into the intracellular expression vector, but it remains to be seen whether this provides any advantage over the well-established scFv format. In addition to scFv and Fab, bispecific formats have been used as intrabodies. A bispecific Tie-2×VEGFR-2 antibody targeted to the ER demonstrated an extended half-life compared to the monospecific antibody counterparts. A bispecific transmembrane intrabody has been developed as a special format to simultaneously recognize intra- and extracellular epitopes of the epidermal growth factor, combining the distinct features of the related monospecific antibodies, i.e., inhibition of autophosphorylation and ligand binding.

Another intrabody format particularly suitable for cytoplasmic expression are single domain antibodies (also called nanobodies) derived from camels or consisting of one human VH domain or human VL domain. These single domain antibodies often have advantageous properties, e.g., high stability; good solubility; ease of library cloning and selection; high expression yield in E. coli and yeast.

The intrabody gene can be expressed inside the target cell after transfection with an expression plasmid or viral transduction with a recombinant virus. Typically, the choice is aimed at providing optimal intrabody transfection and production levels. Successful transfection and subsequent intrabody production can be analyzed by immunoblot detection of the produced antibody, but, for the evaluation of correct intrabody/antigen-interaction, co-immunoprecipitation from HEK 293 cell extracts transiently cotransfected with the corresponding antigen and intrabody expression plasmids may be used.

As used herein, inhibition of SETDB1 gene expression includes any decrease in expression or protein activity or level of the SETDB1 gene or protein encoded by said SETDB1 gene as compared to a situation wherein no inhibition has been induced. The decrease can be of at least, 20%, 30%, 40%, 50%, 60%, 70%, 80%, 90%, 95%, 99% as compared to the expression of SETDB1 gene or level of the SETDB1 protein which has not been targeted by inhibition. Inhibitors of H3K9 histone methyl transferase SETDB1 gene expression can also be selected from anti-sense oligonucleotide constructs, siRNAs, shRNAs, micro RNA (miRNA) and ribozymes.

Anti-sense oligonucleotides, including anti-sense RNA molecules and anti-sense DNA molecules, would act to directly block the translation of H3K9-histone methyltransferase SETDB1 and thus preventing protein translation or increasing mRNA degradation, thus decreasing the level of H3K9-histone methyltransferase SETDB1 and thus its activity in a cell. For example, antisense oligonucleotides of at least about 15 bases and complementary to unique regions of the mRNA transcript sequence encoding H3K9-histone methyltransferase SETDB1 can be synthesized, e.g., by conventional phosphodiester techniques and administered by e.g., intravenous injection or infusion. Methods for using antisense techniques for specifically inhibiting gene expression of genes whose sequence is known are well known in the art (see for example U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of expression for use in the present invention. SETDB1 gene expression can be reduced by contacting a subject or cell with a small double stranded RNA (dsRNA), or a vector or construct causing the production of a small double stranded RNA, such that SETDB1-histone methyltransferase gene expression is specifically inhibited (i.e. RNA interference or RNAi). Methods for selecting an appropriate dsRNA or dsRNA-encoding vector are well known in the art for genes whose sequence is known (see for example Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836). All or parts of the phosphodiester bonds of the siRNAs of the invention are advantageously protected. This protection is generally implemented via the chemical route using methods that are known in the art. The phosphodiester bonds can be protected, for example, by a thiol or amine functional group or by a phenyl group. The 5′- and/or 3′-ends of the siRNAs of the invention are also advantageously protected, for example, using the technique described above for protecting the phosphodiester bonds. The siRNA sequences advantageously comprise at least twelve contiguous dinucleotides or their derivatives.

As used herein, the term “siRNA derivatives” with respect to the present nucleic acid sequences refers to any nucleic acid having a percentage of identity of at least 90% with erythropoietin or fragment thereof, preferably of at least 95%, as an example of at least 98%, and more preferably of at least 98%.

As used herein, the expression “percentage of identity” between two nucleic acid sequences, means the percentage of identical nucleic acid, between the two sequences to be compared, obtained with the best alignment of said sequences, this percentage being purely statistical and the differences between these two sequences being randomly spread over the nucleic acid acids sequences. As used herein, “best alignment” or “optimal alignment”, means the alignment for which the determined percentage of identity (see below) is the highest. Sequence comparison between two nucleic acids sequences is usually realized by comparing these sequences that have been previously aligned according to the best alignment; this comparison is realized on segments of comparison in order to identify and compare the local regions of similarity. The best sequences alignment to perform comparison can be realized, besides manually, by using the global homology algorithm developed by SMITH and WATERMAN (Ad. App. Math., vol. 2, p:482, 1981), by using the local homology algorithm developed by NEDDLEMAN and WUNSCH (J. Mol. Biol, vol. 48, p:443, 1970), by using the method of similarities developed by PEARSON and LIPMAN (Proc. Natl. Acd. Sci. USA, vol. 85, p:2444, 1988), by using computer softwares using such algorithms (GAP, BESTFIT, BLAST P, BLAST N, FASTA, TFASTA in the Wisconsin Genetics software Package, Genetics Computer Group, 575 Science Dr., Madison, Wis. USA), by using the MUSCLE multiple alignment algorithms (Edgar, Robert C, Nucleic Acids Research, vol. 32, p: 1792, 2004). To get the best local alignment, one can preferably use BLAST software. The identity percentage between two sequences of nucleic acids is determined by comparing these two sequences optimally aligned, the nucleic acids sequences being able to comprise additions or deletions in respect to the reference sequence in order to get the optimal alignment between these two sequences. The percentage of identity is calculated by determining the number of identical positions between these two sequences, and dividing this number by the total number of compared positions, and by multiplying the result obtained by 100 to get the percentage of identity between these two sequences.

shRNAs (short hairpin RNA) can also function as inhibitors of expression for use in the present invention.

MicroRNAs (miRNAs) are small (about 21-23 nucleotides) noncoding RNAs that post transcriptionally regulating target gene expression through base pairing to partially complementary sites to prevent protein accumulation by repressing translation or by inducing mRNA degradation. These characteristics make them a possible tool for inhibiting protein translation. As per the invention miRNA can be selected from miR7 and miR9 (Juanjuan Zhao et al., “MicroRNA-7: a promising new target in cancer therapy” Cancer Cell International 2015; 15:103; Zhang H et al., “MiR-7, inhibited indirectly by lincRNA HOTAIR, directly inhibits SETDB1 and “reverses the EMT of breast cancer stem cells by downregulating the STAT3 pathway.” Stem Cells. 2014 November; 32(11):2858-68 and see also Archana Venkataramana Karanth et al., “Emerging role of SETDB1 as a therapeutic target” Expert Opinion on Therapeutics targets 2017).

Ribozymes can also function as inhibitors of expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of H3K9-histone methyltransferase SETDB1 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable.

Both antisense oligonucleotides and ribozymes useful as inhibitors of expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-0-methyl rather than phosphodiesterase linkages within the oligonucleotide backbone.

Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and preferably cells expressing H3K9-histone methyltransferase SETDB1. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and R A virus such as a retrovirus. One can readily employ other vectors not named but known in the art.

Preferred viral vectors are based on non-cytopathic eukaryotic viruses in which nonessential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991).

Preferred viruses for certain applications are the adenoviruses and adeno-associated (AAV) viruses, which are double-stranded DNA viruses that have already been approved for human use in gene therapy. Currently, 12 different AAV serotypes (AAV1 to 12) are known, each with different tissue tropisms (Wu, Z Mol Ther 2006; 14:316-27). Recombinant AAVs are derived from the dependent parvovirus AAV2 (Choi, V W J Virol 2005; 79:6801-07). The adeno-associated virus type 1 to 12 can be engineered to be replication deficient and is capable of infecting a wide range of cell types and species (Wu, Z Mol Ther 2006; 14:316-27). It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate delivery vehicles and micro encapsulation.

The antisense oligonucleotide, siRNA, shRNA or ribozyme nucleic acid sequence according to the invention is generally under the control of a heterologous regulatory region, e.g., a heterologous promoter. The promoter may be specific for Muller glial cells, microglia cells, endothelial cells, pericyte cells and astrocytes For example, a specific expression in Muller glial cells may be obtained through the promoter of the glutamine synthetase gene is suitable. The promoter can also be, as a matter of example, a viral promoter, such as CMV promoter or any synthetic promoters.

In the context of the present invention, an inhibitor of H3K9-histone methyltransferase SETDB1 according to the present invention is preferably selective for H3K9-histone methyltransferase SETDB1, as compared with other histone methyltransferases such EZH2, G9A Suv39H1 or Suv39H2. By “selective” it is meant that the affinity of the inhibitor is at least 10-fold, preferably 25-fold, more preferably 100-fold, and still preferably 500-fold higher than the affinity for other histone methyltransferases.

Typically, the inhibitor of SETDB1 of the invention has an IC₅₀ of less than 20 μM, preferably less than 10 μM, more preferably less than 5 μM, even more preferably less than 1 μM and notably less than 0.5 μM or less 0.1 μM. Typically also the inhibitor of SETDB1 has an IC₅₀ for the other methyltransferases (such as for example EZH2, G9A, Suv39H1 or Suv39H2), notably for other H3k9 methyltransferases, of more than 5 μM, notably more than 10 μM, more than 20 μM, and even more preferably more than 50 μM. For example, an inhibitor of the invention may exhibit an ID50 of less than 1 μM, notably less than 0.5 μM for SETDB1 and more than 10 μM, notably more than 200 for the other methyltransferase (such as example EZH2, G9A, Suv39H1 or Suv39H2), and in particular for H3K9 methyltransferases.

Preferably, the inhibitor of SETDB1 according to the present invention is not selected from triptolide, chaetocin, and verticillin A.

### Immune Checkpoint Modulators

As used herein the term “immune checkpoint protein” (also named immune checkpoint molecule) has its general meaning in the art and refers to a molecule that is expressed by T cells and/or by NK cells and that either turn up a signal (stimulatory checkpoint molecules) or turn down a signal (inhibitory checkpoint molecules). Most preferably according to the invention the immune checkpoint molecule is at least expressed by T cells.

Immune checkpoint molecules are recognized in the art to constitute immune checkpoint pathways similar to the CTLA-4 and PD-1 dependent pathways. Immune checkpoint molecules according to the invention are notably described in Pardoll, 2012. Nature Rev Cancer 12:252-264; Mellman et al., 2011. Nature 480:480-489; Chen L & Flies D B, Nat. Rev. Immunol. 2013 April; 13(4):227-242, and Kemal Catakovic, Eckhard Klieser et al., “T cell exhaustion: from pathophysiological basics to tumor immunotherapy” Cell Communication and Signaling 2017, 15:1). Example of immune checkpoints molecules notably encompasses CD27, CD40, OX40, GITR, ICOS, TNFRSF25, 41BB, HVEM, CD28, TMIGD2, CD226, 2B4 (CD244) and ligand CD48, B7-H6 Brandt (NK ligand), LIGHT (CD258, TNFSF14), CD28H, A2AR, B7-H3, B7-H4, BTLA, CTLA-4, CD277, IDO, KIRs, PD-1s, LAG-3, TIM-3 TIGIT, VISTA, CD96, CD112R, CD160, CD244 (or 2B4) DCIR (C-type lectin surface receptor), ILT3, ILT4 (Immunoglobulin-like transcript), CD31 (PECAM-1) (Ig-like R family), CD39, CD73, CD94/NKG2, GP49b (immunoglobulin superfamily), KLRG1, LAIR-1 (Leukocyte-associated immunoglobulin-like receptor 1) CD305, PD-L1 and PD-L2 and SIRPα.

Non-limitative examples of inhibitory checkpoint molecules include A2AR, B7-H3, B7-H4, BTLA, CTLA-4, CD277, IDO, KIRs, PD-1, LAG-3, TIM-3 TIGIT, VISTA, CD96, CD112R, CD160, DCIR (C-type lectin surface receptor), ILT3, ILT4 (Immunoglobulin-like transcript), CD31 (PECAM-1) (Ig-like R family), CD39, CD73, CD94/NKG2, GP49b (immunoglobulin superfamily), KLRG1, LAIR-1 (Leukocyte-associated immunoglobulin-like receptor 1), CD305, PD-L1 and PD-L2.

The Adenosine A2a receptor (A2aR), the ligand of which is adenosine, is regarded as an important checkpoint in cancer therapy because adenosine in the immune microenvironment, leading to the activation of the A2a receptor, is negative immune feedback loop and the tumor microenvironment has relatively high concentrations of adenosine. A2aR can be inhibited by antibodies that block adenosine binding or by adenosine analogues some of which are fairly specific for A2aR. These drugs have been used in clinical trials for Parkinson's disease.

The B7 family is an important family of membrane-bound ligand that binds co-stimulatory and inhibitory receptors. All of the B7 family members and their known ligands belong to the immunoglobulin superfamily. Many receptors have not been yet identified. B7-H3, also called CD276, was originally understood to be a co-stimulatory molecule but is now regarded as co-inhibitory. B7-H4, also called VTCN1, is expressed by tumor cells and tumor-associated macrophages and plays a role in tumor escape.

CD160 is a glycosylphosphatidylinositol (GPI)-anchored protein member of the Ig superfamily with a restricted expression profile that is limited to CD56dim CD16+ NK cells, NKT-cells, γδ T-cells, cytotoxic CD8+ T-cells lacking the expression of CD28, a small fraction of CD4+ T cells and all intraepithelial lymphocytes. Binding of CD160 to both classical and non-classical MHC I enhances NK and CD8+ CTL functions. However, engagement of CD160 by the Herpes Virus Entry Mediator (HVEM/TNFRSF14) was shown to mediate inhibition of CD4+ T-cell proliferation and TCR-mediated signaling.

HVEM (Herpesvirus Entry Mediator) protein is a bimolecular switch that binds both co-stimulatory LT-a/LIGHT and co-inhibitory receptors BTLA/CD160. The ligation of coinhibitory receptors BTLA and/or CD160 on T cells with HVEM expressed on DC or Tregs transduces negative signals into T cells that are counterbalanced by costimulatory signals delivered after direct engagement of HVEM on T cells by LIGHT expressed on DC or more likely, on other activated T cells (T-T cell cooperation). The predominance of the interaction of HVEM with BTLA and CD160 over the HVEM/LIGHT pathway or vice versa might be the result of differences in ligand/receptor affinity and the differential expression pattern of these molecules on cell types at different stages of cell differentiation. LIGHT, BTLA, and CD160 have substantially different binding affinities and occupy spatially distinct sites upon interaction with the HVEM receptor, which enables HVEM to function as a molecular switch. The net effect of the LIGHT/HVEM and HVEM/BTLA/CD160 interaction, when these different receptors and ligands are simultaneously present, determines the outcome of the response (see M. L. del Rio. “HVEM/LIGHT/BTLA/CD160 cosignaling pathways as targets for immune regulation” Journal of Leukocyte Biology. 2010; 87).

B and T Lymphocyte Attenuator (BTLA), also called CD272, has also HVEM as its ligand. BTLA T cells are inhibited in the presence of its ligand, HVEM. Surface expression of BTLA is gradually downregulated during differentiation of human CD8+ T cells from the naive to effector cell phenotype, however tumor-specific human CD8+ T cells express high levels of BTLA (Kenneth M. Murphy et al. Balancing co-stimulation and inhibition with BTLA and HVEM. Nature Reviews Immunology 2006, 6, 671-681).

CTLA-4, Cytotoxic T-Lymphocyte-Associated protein 4 also called CD152, was the first immune checkpoint to be clinically targeted. It is expressed exclusively on T cells. It has been proposed that its expression on the surface of T cells dampens the activation of T cells by outcompeting CD28 in binding CD80 and CD86 as well as actively delivering inhibitory signals to the T cells. Expression of CTLA-4 on Treg cells serves to control T cell proliferation.

Ig-like transcript-3 and -4 (ILT3 and ILT4) are inhibitory receptors both expressed by monocytes, macrophages, and DCs. The corresponding ILT3 ligand is not yet known, but since ILT3 can directly suppress T lymphocyte function, it is likely to be expressed on T cells. In several cancers, ILT3 has been found to mediate the immune escape mechanism by impairing T cell responses. Furthermore, ILT4-expressing DCs block efficient CTL differentiation, a mechanism that is used by tumors, which upregulate ILT4 to evade the immune system (Vasaturo A et al., Front Immunol. 2013; 4: 417).

Platelet endothelial cell adhesion molecule-1 (PECAM-1), also known as CD31, is a type I transmembrane glycoprotein member of the immunoglobulin (Ig) gene superfamily which contains six extracellular Ig domains and two cytoplasmic immunoreceptor tyrosine-based inhibitory motifs (ITIMs). PECAM-1 is restricted to endothelial cells and cells of the hematopoietic system (see Newman D K, Fu G, Adams T, et al. The adhesion molecule PECAM-1 enhances the TGFβ-mediated inhibition of T cell function. Science signaling. 2016; 9(418):ra27).

LAIR-1 is expressed in very high and relatively homogenous levels in naive T cells but in lower and more heterogeneous levels in memory T cells. LAIR-1 consist of a type I transmembrane glycoprotein of 287 amino acids with a single extracellular C2-type Iglike domain and a cytoplasmic domain with two ITIM motifs. LAIR-1 can inhibit TCR mediated signals possibly through the recruitment of C-terminal Csk, one or more of the phosphatases SHIP, SHP-1 or SHP-2, and to a certain extent on signaling through p38 MAP kinase and ERK signaling (Thaventhiran T et al. (2012) J Clin Cell Immunol S12:004).

IDO1, Indoleamine 2,3-dioxygenase 1, is a tryptophan catabolic enzyme. A related immune-inhibitory enzymes. Another important molecule is TDO, tryptophan 2,3-dioxygenase. IDO1 is known to suppress T and NK cells, generate and activate Tregs and myeloid-derived suppressor cells, and promote tumor angiogenesis.

KIR, Killer-cell Immunoglobulin-like Receptor, are a braid category of inhibitory receptors that can be divided into two classes based on structure: killer cell immunoglobulin-like receptors (KIRs) and C-type lectin receptors which are type II transmembrane receptors. There receptors where originally described as regulators of the killing activity of NK cells although many are expressed on T cells and APCs. Many if the KIRs are soecufuc for subsets MHC class I molecules and possess allele-specificity.

LAG3, Lymphocyte Activation Gene-3 has, as its ligand, MHC class II molecules, which are upregulated on some epithelial cancers but are also expressed on tumor-infiltrating macrophages and dendritic cells. This immune checkpoint works to suppress an immune response by action to T_{reg} cells as well as direct effects on CD8+ T cells.

PD-1, Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment.

TIM-3 short for T-cell Immunoglobulin domain and Mucin domain 3 (also named B7H5), and the ligand of which is galacting 9, is expressed on activated human CD4+ T cells and regulates Th1 and Th17 cytokines. TIM-3 acts as a negative regulator of Th1/Tc1 function by triggering cell death upon interaction with its ligand, galectin-9.

VISTA (short for V-domain Ig suppressor of T cell activation) VISTA, also known as c10orf54, PD-1H, DD1α, Gi24, Dies1, and SISP1] is a member of the B7 family of NCRs and represents a new target for immunotherapy. Murine VISTA is a type I transmembrane protein with a single IgV domain with sequence homology to its B7 relatives with conserved segments thought to be critical for the IgV stability. VISTA is expressed on naïve T cells whereas PD-1 and CTLA-4 are not, which may suggest that VISTA functions to restrain T cell activity at an even earlier stage in T cell priming. VISTA is expressed on both T cells and APCs with very high expression on myeloid cells. VISTA is hematopoietically restricted and in multiple cancer models, VISTA was only detected on tumor infiltrating leukocytes and not on tumor cells. This unique surface expression pattern suggests that VISTA may function to restrict T cell immunity at different stages. VISTA has been demonstrated to exert both ligand and receptor functions. First, VISTA can function as a ligand to negatively regulate T cell activation. Second, VISTA has been demonstrated to function as a receptor on T cells which negatively regulates their activity. VISTA^{-/-} CD4⁺ T cells respond more vigorously than wild type (WT) CD4⁺ T cells to both polyclonal and antigen specific stimulation leading to increased proliferation and production of IFNγ, TNFα, and IL-17A. Anti-VISTA monotherapy reduced tumor growth in multiple pre-clinical models, B16OVA melanoma, B16-BL6 melanoma, MB49 bladder carcinoma, and PTEN/BRAF inducible melanoma (see Deng J, Le Mercier I, Kuta A, Noelle R J. “A New VISTA on combination therapy for negative checkpoint regulator blockade. J Immunother Cancer. 2016 Dec. 20; 4:86. doi: 10.1186/s40425-016-0190-5. eCollection 2016. Review; see also Kathleen M. Mahoney et al., “Combination cancer immunotherapy and new immunomodulatory targets”. Nature Reviews Drug Discovery 2015; 14:561-584).

CD96, CD226 (DNAM-1) and TIGIT belong to an emerging family of receptors that interact with nectin and nectin-like proteins. CD226 activates natural killer (NK) cell-mediated cytotoxicity, whereas TIGIT reportedly counterbalances CD226.

CD96 competes with CD226 for CD155 binding and limites NK cell function by direct inhibition (Christopher J Chan et al., “The receptors CD96 and CD226 oppose each other in the regulation of natural killer cell functions”, Nature Immunology 2014 15, 431-438).

TIGIT (also called T cell immunoreceptor with Ig and ITIM domains, or VSTM3) TIGIT/VSTM3 is expressed normally by activated T cells, regulatory T (Tᵣₑₛ) cells, and natural killer (NK) cells. The poliovirus receptor (CD155/PVR) and Nectin-2 (CD112) as well as CD 113 have been identified as relevant ligands. TIGIT/VSTM3 competes with the molecules CD226 and CD96 for binding to CD155/PVR and CD112, respectively, but among all respective receptor-ligand combinations, TIGIT/VSTM3 exhibits the strongest affinity for CD155/PVR. TIGIT inhibits T cell activation in vivo (see Karsten Mahnke et al. TIGIT-CD155 Interactions in Melanoma: A Novel Co-Inhibitory Pathway with Potential for Clinical Intervention. Journal of Investigative Dermatology. 2016; 136: 9-11).

CD112R (PVRIG), the ligand of which is PVRL2, is a member of poliovirus receptor-like proteins which is preferentially expressed on T cells and inhibits T cell receptor-mediated signals.

Non-limitative examples of stimulatory checkpoint molecules include CD27, CD40L, OX40, GITR, ICOS, TNFRSF25, 41BB, HVEM, CD28, TMIGD2, and CD226, 2B4 (CD244) and its ligand CD48, B7-H6 Brandt (NK ligand), CD28H and LIGHT (CD258, TNFSF14).

CD27, CD40L, OX40, GITR, ICOS, HVEM, 2B4 (CD244) and its ligand CD48, B7-H6 Brandt (NK ligand), LIGHT (CD258, TNFSF14), CD28H and TNFSF25 are stimulatory checkpoint molecules, which are members of the tumor necrosis factor (TNF) receptor superfamily (TNFSF). TNFRSF proteins play an important role in B and T cell development, survival, and antitumor immune response. In addition, some TNFRSFs are involved in the deactivation of T_{reg} cells. Therefore, TNFRSF agonists activate tumor immunity, and their combination with immune checkpoint therapy is promising. Several antibodies that act as TNFRSF agonist have been evaluated in clinical trials (Shiro Kimbara and Shunsuke Kondo, “Immune checkpoint and inflammation as therapeutic targets in pancreatic carcinoma”, World J Gastroenterol. 2016 Sep. 7; 22(33): 7440-7452, see also for review Watts T H. TNF/TNFR family members in costimulation of T cell responses. Annu Rev Immunol. 2005; 23:23-68.).

CD27 supports antigen-specific expansion of naïve T cells and is vital for the generation of T cell memory. CD27 is also a memory marker of B cells. CD27's activity is governed by the transient availability of its ligand, CD70, on lymphocytes and dendritic cells. CD27 costimulation is known to suppresses Th17 effector cell function

The CD40:CD40L pathway is a co-stimulatory pathway that affects both humoral and cell-mediated immunity. CD40L (also known as CD154), is primarily expressed on T-helper cells shortly after activation. The receptor 2B4 (CD244) belongs to the signaling lymphocyte activation molecule (SLAM) subfamily within the immunoglobulin superfamily (IgSV). All members of this family contain two or more immunoreceptor tyrosine-based switch motifs (ITSMs) in their cytoplasmatic tail including the receptors CD229, CS1, NTB-A and CD84 [92]. 2B4 is expressed by NK cells, yb T cells basophils and monocytes, upon activation on CD8+ T cells and binds with high affinity to CD48 on lymphoid and myeloid cells (Kemal Catakovic et al., Cell Communication and Signaling201715:1).

TNFSF14/LIGHT/CD258 exhibits inducible expression, and competes with herpes simplex virus (HSV) glycoprotein D for herpesvirus entry mediator (HVEM/TNFRSF14), a receptor expressed by T lymphocytes, is a recently identified member of the human and mouse TNF superfamily. TNFSF14/LIGHT/CD258 is a 29-kD type II transmembrane protein produced by activated T cells, as well as monocytes and granulocytes, and immature DCs. In vitro, HVEM/LIGHT immune checkpoint pathway induces potent CD28-independent costimulatory activity, leading to NF-κB activation, production of IFN-γ and other cytokines, and T cell proliferation in response to allogeneic DCs. In vivo blockade studies show HVEM/LIGHT immune checkpoint pathway is involved in promotion of cytolytic T cell responses to tumors and the development of GVHD, and transgenic overexpression of TNFSF14/LIGHT/CD258 within T cells leads to T cell expansion and causes various severe autoimmune diseases (Qunrui Ye et al. J Exp Med. 2002 Mar. 18; 195(6): 795-800).

CD28H is constitutively expressed on all naive T cells. B7 homologue 5 (B7-H5), was identified as a specific ligand for CD28H. B7-H5 is constitutively found in macrophages and could be induced on dendritic cells. The B7-H5/CD28H interaction selectively costimulates human T-cell growth and cytokine production via an AKT-dependent signalling cascade (Zhu Y et al., Nat Commun. 2013; 4:204).

OX40, also called CD134, has OX40L, or CD252, as its ligand. Like CD27, OX40 promotes the expansion of effector and memory T cells, however it is also noted for its ability to suppress the differentiation and activity of T-regulatory cells, and also for its regulation of cytokine production. OX40's value as a drug target primarily lies it the fact that, being transiently expressed after T-cell receptor engagement, it is only upregulated on the most recently antigen-activated T cells within inflammatory lesions. Anti-OX40 monoclonal antibodies have been shown to have clinical utility in advanced cancer (Weinberg A D, Morris N P, Kovacsovics-Bankowski M, Urba W J, Curti B D (Nov. 1, 2011). “Science gone translational: the OX40 agonist story”. Immunol Rev. 244 (1): 218-31).

GITR, short for Glucocorticoid-Induced TNFR family Related gene, prompts T cell expansion, including Treg expansion. The ligand for GITR (GITRL) is mainly expressed on antigen presenting cells. Antibodies to GITR have been shown to promote an anti-tumor response through loss of T_{reg} lineage stability (see Nocentini G, Ronchetti S, Cuzzocrea S, Riccardi C (May 1, 2007). “GITR/GITRL: more than an effector T cell co-stimulatory system”. Eur J Immunol. 37 (5): 1165-9).

ICOS, short for Inducible T-cell costimulator, and also called CD278, is expressed on activated T cells. Its ligand is ICOSL, expressed mainly on B cells and dendritic cells.

The molecule seems to be important in T cell effector function (Burmeister Y, Lischke T, Dahler A C, Mages H W, Lam K P, Coyle A J, Kroczek R A, Hutloff A (Jan. 15, 2008). “ICOS controls the pool size of effector-memory and regulatory T cells”. J Immunol. 180 (2): 774-782).

Another stimulatory checkpoint molecules, which belongs to the B7-CD28 superfamily, are notably CD28 itself and TGMID2.

CD28 is constitutively expressed on almost all human CD4+ T cells and on around half of all CD8 T cells. Binding with its two ligands (CD80 and CD86, expressed on dendritic cells) prompts T cell expansion.

TMIGD2 (also called CD28 homolog), modulates T cell functions through interaction with its ligand HHLA2; a newly identified B7 family member. TMIGD2 protein is constitutively expressed on all naïve T cells and the majority of natural killer (NK) cells, but not on T regulatory cells or B cells (see Yanping Xiao and Gordon J. Freeman, “A new B7:CD28 family checkpoint target for cancer immunotherapy: HHLA2”, Clin Cancer Res. 2015 May 15; 21(10): 2201-2203).

CD137 ligand (CD137L; also known as 4-1BBL and TNFSF9) is mainly expressed on professional antigen-presenting cells (APCs) such as dendritic cells, monocytes/macrophages, and B cells, and its expression is upregulated during activation of these cells. However, its expression has been documented on a variety of hematopoietic cells and nonhematopoietic cells. Generally, 4-1BBL/CD137L is constitutively expressed on many types of cells but its expression levels are low except for a few types of cells. Interestingly, 4-1BBL/CD137L is coexpressed with CD137 (also known as 4-1 BB and TNFRSF9) on various types of cells, but expression of CD137/4-1BB potently downregulates that of 4-1BBL/CD137L by cis-interactions between the two molecules resulting in endocytosis of 4-1 BBL/CD137L (see Byungsuk Kwon et al. Is CD137 Ligand (CD137L) “Signaling a Fine Tuner of Immune Responses?” Immune Netw. 2015 June; 15(3):121-124).

Finally other immune checkpoint molecules according to the invention also include CD244 (or 2B4) and SIRPα.

2B4/CD244 is a member of the signaling lymphocyte activation molecule (SLAM)-related receptor family and is also known as SLAMF4 and CD244. All members of the SLAM family share a similar structure, including an extracellular domain, a transmembrane region, and a tyrosine rich cytoplasmic region. 2B4 & CD48 Immune Checkpoint Pathway can lead to signaling through both receptors. CD48/SLAMF2 signaling in B cells leads to homotypic adhesion, proliferation and/or differentiation, release of inflammatory effector molecules and isotype class switching. In addition, all of these processes are also elicited in T cells via CD48/SLAMF2 ligation with the addition of promoting their activation and/or cytotoxicity. 2B4 signaling requires signaling lymphocyte activation molecule (SLAM)-associated protein (SAP) or EWS-activated transcript 2 (EAT-2; also called SH2D1B). In CD8 T cells and NK cells 2B4/CD244 has been reported to exert both positive and negative regulation (see also Sebastian Stark. “2B4 (CD244), NTB-A and CRACC (CS1) stimulate cytotoxicity but no proliferation in human NK cells”. Int. Immunol. 2006 18 (2): 241-247).

CD47 is a cell surface glycoprotein with a variety of functions including regulation of phagocytosis through binding to the macrophage and dendritic cell specific protein signal regulatory protein alpha (SIRP alpha). Binding of SIRP alpha to CD47, as SIRP alpha & CD47 immune checkpoint pathway, essentially sends a “don't eat me” message to macrophages by initiating signaling to inhibit phagocytosis. Increased expression of CD47 is proposed to be a mechanism through which cancer cells evade immune detection and phagocytosis. Targeting of CD47 on cancer cells with an anti-CD47 blocking antibody can promote phagocytosis by macrophages in vitro. Further, treatment with an anti-CD47 blocking antibody synergized with rituximab treatment to promote phagocytosis in vitro and to eliminate cancer cells in an in vivo xenograft model of non-Hodgkin lymphoma. Further results demonstrate that CD47 expression increases in a variety of human solid tumor types and that blocking the SIRP alpha & CD47 immune checkpoint pathway with an anti-CD47 antibody can promote phagocytosis of solid tumor cells in vitro and reduce growth of solid tumors in vivo (see Martina Seiffert et al. “Signal-regulatory protein a (SIRPα) but not SIRPβ is involved in T-cell activation, binds to CD47 with high affinity, and is expressed on immature CD34+CD38−hematopoietic cells”. 2001; Blood: 97 (9)).

As used herein, the expression “modulator of an immune checkpoint protein”, or “checkpoint regulator cancer immunotherapy agent” (both expressions can be used interchangeably in the sense of the invention) has its general meaning in the art and refers to any compound inhibiting the function of an immune inhibitory checkpoint protein (inhibitory immune checkpoint inhibitors, or immune checkpoint inhibitors as previously described) or stimulating the function of a stimulatory checkpoint protein (stimulatory immune checkpoint agonist or immune checkpoint agonist used interchangeably). Inhibition includes reduction of function and full blockade.

The immune checkpoint modulators include peptides, antibodies, fusion proteins, nucleic acid molecules and small molecules. For certain immune checkpoint protein (i.e., immune pathway gene products), the use of either antagonists or agonists of such gene products is also contemplated, as are small molecule modulators of such gene products.

Preferred immune checkpoint inhibitors or agonists are antibodies, or fusions proteins that specifically recognize immune checkpoint proteins or their ligands, as described previously.

According to the invention various mixtures of antibodies against either different epitopes of the same molecule or different targets on the same tumor cell; bispecific or multispecific antibodies could be used (Corraliza-Gorjón I, Somovilla-Crespo B, Santamaria S, Garcia-Sanz J A, Kremer L. New Strategies Using Antibody Combinations to Increase Cancer Treatment Effectiveness. Frontiers in Immunology. 2017; 8:1804; Liu H, Saxena A, Sidhu S S, Wu D. Fc Engineering for Developing Therapeutic Bispecific Antibodies and Novel Scaffolds. Front Immunol. 2017 Jan. 26; 8:38. doi: 10.3389/fimmu.2017.00038. eCollection 2017. Review.).

A fusion protein for use as immune checkpoint modulator can be made by fusion of a checkpoint molecule as described above with the crystallizable fragment (Fc) region of an immunoglobulin. Preferably antibodies are monoclonal antibodies.

A number of immune checkpoint inhibitors and agonists are known in the art and in analogy of these known immune checkpoint protein modulators, alternative immune checkpoint modulators may be developed in the (near) future and be used in combination with an inhibitor of SETDB1 according to the invention.

An immune checkpoint modulator according to the invention results in an activation of the immune system and in particular leads to an amplification of antigen-specific T cell response. In particular, the immune checkpoint modulator of the present invention is administered for enhancing the proliferation, migration, persistence and/or cytoxic activity of CD8+ T cells in the subject and in particular the tumor-infiltrating of CD8+ T cells of the subject. As used herein “CD8+ T cells” has its general meaning in the art and refers to a subset of T cells which express CD8 on their surface. They are MHC class I-restricted, and function as cytotoxic T cells. “CD8+ T cells” are also called CD8+ T cells are called cytotoxic T lymphocytes (CTL), T-killer cell, cytolytic T cells, CD8+ T cells or killer T cells. CD8 antigens are members of the immunoglobulin supergene family and are associative recognition elements in major histocompatibility complex class I-restricted interactions. The ability of the immune checkpoint modulator to enhance T CD8 cell killing activity may be determined by any assay well known in the art. Typically said assay is an in vitro assay wherein CD8+ T cells are brought into contact with target cells (e.g. target cells that are recognized and/or lysed by CD8+ T cells).

For example, the immune checkpoint modulator of the present invention can be selected for the ability to increase specific lysis by CD8+ T cells by more than about 20%, preferably with at least about 30%, at least about 40%, at least about 50%, or more of the specific lysis obtained at the same effector: target cell ratio with CD8+ T cells or CD8 T cell lines that are contacted by the immune checkpoint inhibitor of the present invention, Examples of protocols for classical cytotoxicity assays are conventional.

The at least one immune checkpoint modulator according to the invention can be a modulator of an inhibitory immune checkpoint molecule and/or of a stimulatory immune checkpoint molecule.

For example, the checkpoint regulator cancer immunotherapy agent can be an agent which blocks (an antagonist of) an immunosuppressive receptor (i.e., an inhibitory immune checkpoint) expressed by activated T lymphocytes, such as cytotoxic T lymphocyte-associated protein 4 (CTLA4) and programmed cell death 1 (PDCD1, best known as PD-1), or by NK cells, like various members of the killer cell immunoglobulin-like receptor (KIR) family, or an agent which blocks the principal ligands of these receptors, such as PD-1 ligand CD274 (best known as PD-L1 or B7-H1).

In some embodiments, the checkpoint blockade cancer immunotherapy agent is selected from the group consisting of anti-CTLA4 antibodies, anti-PD1 antibodies, anti-PDL1 antibodies, anti-PDL2 antibodies, anti-TIM-3 antibodies, anti-LAG3 antibodies, anti-IDO1 antibodies, anti-TIGIT antibodies, anti-B7H3 antibodies, anti-B7H4 antibodies, anti-BTLA antibodies, anti-B7H6 antibodies, anti-CD86 antibodies, anti-Gal9 antibodies, anti-HVEM antibodies, anti-CD28 antibodies, anti-A2aR antibodies, anti-CD80 antibodies, anti-KIR(s) antibodies, A2aR drugs (notably adenosine analogs), anti-DCIR (C-type lectin surface receptor) antibodies, anti-ILT3 antibodies, anti-ILT4 antibodies, anti-CD31 (PECAM-1) antibodies, anti-CD39 antibodies, anti-CD73 antibodies, anti-CD94/NKG2 antibodies, anti-GP49b antibodies, anti-KLRG1 antibodies, anti-LAIR-1 antibodies, anti-CD305 antibodies, and their combinations. In certain embodiments, the checkpoint blockade cancer immunotherapy agent is an anti-PD-1 or an anti-PD-L1 antibody.

Examples of anti-CTLA-4 antibodies are described in U.S. Pat. Nos. 5,811,097; 5,811,097; 5,855,887; 6,051,227; 6,207,157; 6,682,736; 6,984,720; and 7,605,238. One anti-CDLA-4 antibody is tremelimumab, (ticilimumab, CP-675,206). In some embodiments, the anti-CTLA-4 antibody is ipilimumab (also known as 10D1, MDX-D010) a fully human monoclonal IgG antibody that binds to CTLA-4.

Examples of PD-1 and PD-L1 antibodies are described in U.S. Pat. Nos. 7,488,802; 7,943,743; 8,008,449; 8,168,757; 8,217,149, and PCT Published Patent Application Nos: WO03042402, WO2008156712, WO2010089411, WO2010036959, WO2011066342, WO2011159877, WO2011082400, and WO2011161699. In some embodiments, the PD-1 blockers include anti-PD-L1 antibodies. In certain other embodiments the PD-1 blockers include anti-PD-1 antibodies and similar binding proteins such as nivolumab (MDX 1106, BMS 936558, ONO 4538), a fully human IgG4 antibody that binds to and blocks the activation of PD-1 by its ligands PD-LI and PD-L2; lambrolizumab (MK-3475 or SCH 900475), a humanized monoclonal IgG4 antibody against PD-1; CT-011 a humanized antibody that binds PD-1; AMP-224 is a fusion protein of B7-DC; an antibody Fc portion; BMS-936559 (MDX-1105-01) for PD-L1 (B7-H1) blockade.

Other immune-checkpoint inhibitors include lymphocyte activation gene-3 (LAG-3) inhibitors, such as IMP321, a soluble Ig fusion protein (Brignone et al., 2007, J. Immunol. 179:4202-4211).

Other immune-checkpoint inhibitors include B7 inhibitors, such as B7-H3 and B7-H4 inhibitors, notably, the anti-B7-H3 antibody MGA271 (Loo et al., 2012, Clin. Cancer Res. July 15 (18) 3834).

Also included are TIM3 (T-cell immunoglobulin domain and mucin domain 3) inhibitors (Fourcade et al., 2010, J. Exp. Med. 207:2175-86 and Sakuishi et al., 2010, J. Exp. Med. 207:2187-94). As used herein, the term “TIM-3” has its general meaning in the art and refers to T cell immunoglobulin and mucin domain-containing molecule 3. Accordingly, the term “TIM-3 inhibitor” as used herein refers to a compound, substance or composition that can inhibit the function of TIM-3. For example, the inhibitor can inhibit the expression or activity of TIM-3, modulate or block the TIM-3 signaling pathway and/or block the binding of TIM-3 to galectin-9, its natural ligand. Antibodies having specificity for TIM-3 are well known in the art and typically those described in WO2011155607, WO2013006490 and WO2010117057.

In some embodiments, the immune checkpoint inhibitor is an Indoleamine 2,3-dioxygenase (IDO) inhibitor, preferably an IDO1 inhibitor. Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. Preferably the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

In some embodiments, the immune checkpoint inhibitor is an anti-TIGIT (T cell immunoglobin and ITIM domain) antibody.

In some embodiments, the immune checkpoint inhibitor is an anti-VISTA antibody, preferably a monoclonal antibody (Lines J L, Sempere L F, Wang L, et al. VISTA is an immune checkpoint molecule for human T cells. Cancer research. 2014; 74(7):1924-1932. doi:10.1158/0008-5472.CAN-13-1504).

In a preferred embodiment, the checkpoint modulator cancer immunotherapy agent is a CTLA4 blocking antibody, such as Ipilimumab, a PD-1 blocking antibody, such as Nivolumab or Pembrolizumab, a PDL-1 blocking antibody or a combination thereof. Typically, the checkpoint modulator cancer immunotherapy agent is a PD-1 blocking antibody, such as Nivolumab or Pembrolizumab, or a PDL-1 blocking antibody.

The checkpoint modulator cancer immunotherapy agent can also be an agent, which activates a stimulatory immune checkpoint receptor expressed by activated T lymphocytes, or by NK cells, or an agent which mimics the principal ligands of these receptors, and results also in the amplification of antigen-specific T cell responses.

Thus, the checkpoint modulator cancer immunotherapy agent can typically be an agonistic antibody, notably a monoclonal agonistic antibody to a stimulatory immune checkpoint molecules as described above, for example selected from the group consisting of agonistic anti-4-1BB, -OX40, -GITR, -CD27, -ICOS, -CD40L, -TMIGD2, -CD226, -TNFSF25, -2B4 (CD244), -CD48, -B7-H6 Brandt (NK ligand), -CD28H-LIGHT (CD258, TNFSF14), and -CD28 antibodies.

The checkpoint agonist cancer immunotherapy agent can also be a fusion protein for example, a 4-1BB-Fc fusion protein, an Ox40-Fc fusion protein, a GITR-Fc fusion protein, a CD27-Fc fusion protein, an ICOS-Fc fusion protein, a CD40L-Fc fusion protein, a TMIGD2-Fc fusion protein, a CD226-Fc fusion protein, a TNFSF25-Fc fusion protein, a CD28-Fc fusion protein, a 2B4 (CD244) fusion protein, a CD48 fusion protein, a B7-H6 Brandt (NK ligand) fusion protein, a CD28H fusion protein and a LIGHT (CD258, TNFSF14) fusion protein.

Several of the 4-1 BB agonists show great potential for application to human cancers. For example, BMS-666513, a fully humanized mAb against 4-1BB, has completed phase I and II trials for its anticancer properties in patients with melanoma, renal cell carcinoma, and ovarian cancer (Sznol M, Hodi F S, Margolin K, McDermott D F, Ernstoff M S, Kirkwood J M, et al. Phase I study of BMS-663513, a fully human anti-CD137 agonist monoclonal antibody, in patients (pts) with advanced cancer (CA). J Clin Oncol 26: 2008 (May 20 suppl; abstr 3007).

Seven OX40 agonists are now in development, 6 of which take the form of fully human monoclonal antibodies to address the mouse antibody issue. One OX40L-Fc fusion protein, MED16383, is also undergoing clinical evaluation; this links 2 OX40L molecules to part of the fragment crystallizable (Fc) region of immunoglobulin. In preclinical testing, the fusion protein appears to have stronger effects than OX40 antibodies, possibly because it may also activate dendritic cells and vascular endothelial cells in addition to T cells. Examples of Ox40 agonists include MEDI6469, MED16383, MED10652, PF-04515600, MOXP0916, GSK3174998, INCAGNO 1949.

Agonistic antibodies to GITR have been developed such as a humanized anti-human GITR mAb (TRX518. Tolerx Inc. Agonistic antibodies to human glucocorticoid-induced tumor necrosis factor receptor as potential stimulators of T cell immunity for the treatment of cancer and viral infections. Expert Opin Ther Patents. 2007; 17:567-575, see also Schaer D A, Murphy J T, Wolchok J D. Modulation of GITR for cancer immunotherapy. Curr Opin Immunol. 2012 April; 24(2):217-24).

An example of an agonistic antibody to CD27, another member of the TNF family include the fully human 1 F5 mAb that is now in Phase I clinical testing in B-cell malignancies, melanoma and renal cell carcinoma as CDX-1127 (varlilumab) (Analysis of the properties of the anti-CD27 monoclonal antibody (mAb) that is currently in clinical trials (Vitale L A, He L-Z, Thomas L J et al. 2012 Development of a human monoclonal antibody for potential therapy of CD27-expressing lymphoma and leukemia. Clin. Cancer Res. 18(14), 3812-3821).

Initial clinical trials of agonistic CD40 mAb have shown highly promising results in the absence of disabling toxicity, in single-agent studies. To date, four CD40 mAb have been investigated in clinical trials: CP-870,893 (Pfizer and VLST), dacetuzumab (Seattle Genetics), Chi Lob 7/4 (University of Southampton), and lucatumumab (Novartis) (Vonderheide R H, Flaherty K T, Khalil M, Stumacher M S, Bajor D L, Hutnick N A, et al. Clinical activity and immune modulation in cancer patients treated with CP-870,893, a novel CD40 agonist monoclonal antibody. J Clin Oncol. 2007; 25:876-83; Khubchandani S, Czuczman M S, Hernandez-Ilizaliturri F J. Dacetuzumab, a humanized mAb against CD40 for the treatment of hematological malignancies. Curr Opin Investig Drugs. 2009; 10:579-87; Johnson P W, Steven N M, Chowdhury F, Dobbyn J, Hall E, Ashton-Key M, et al. A Cancer Research UK phase I study evaluating safety, tolerability, and biological effects of chimeric anti-CD40 monoclonal antibody (MAb), Chi Lob 7/4. J Clin Oncol. 2010; 28:2507; Bensinger W, Maziarz R T, Jagannath S, Spencer A, Durrant S, Becker P S, et al. A phase 1 study of lucatumumab, a fully human anti-CD40 antagonist monoclonal antibody administered intravenously to patients with relapsed or refractory multiple myeloma. Br J Haematol. 2012; 159:58-66).

The checkpoint agonist cancer immunotherapy agent can also be an anti-ICOS agonist monoclonal antibody (Kutlu Elpek, Christopher Harvey, Ellen Duong, Tyler Simpson, Jenny Shu, Lindsey Shallberg, Matt Wallace, Sriram Sathy, Robert Mabry, Jennifer Michaelson, and Michael Briskin, Abstract A059: Efficacy of anti-ICOS agonist monoclonal antibodies in preclinical tumor models provides a rationale for clinical development as cancer immunotherapeutics; Abstracts: CRI-CIMT-EATI-AACR Inaugural International Cancer Immunotherapy Conference: Translating Science into Survival; Sep. 16-19, 2015; New York, N.Y.), or an anti-CD28 agonist antibody (for use notably in combination with anti-PD-1 immunotherapy, see T cell costimulatory receptor CD28 is a primary target for PD-1-mediated inhibition) see also Melero I, Hervas-Stubbs S, Glennie M, Pardoll D M, Chen L. Nat Rev Cancer. 2007 February; 7(2):95-106, for review.

According to the present invention more than one modulator of an immune checkpoint protein can be used in combination with the inhibitor of SETDB1 according to the present invention. For example, at least one modulator of an inhibitory immune checkpoint inhibitor (such as an anti-PD-1 or an anti-PD-L1) can be used in combination with at least one stimulatory immune checkpoint agonist as mentioned above. Co-stimulatory and co-inhibitory immune checkpoint molecules are notably described in the review of Chen L & Flies B (Nat rev Immuno., 2013 mentioned above).

### Patients

Typically, the patient according to the invention is a mammalian, preferably a human.

Typically said patient is suffering from a cancer, or is in remission or is at risk of a cancer. A patient in remission is typically a patient, wherein the cancer has been treated (for example by surgery removal) and is no longer present. Thus typically the combination treatment of the present invention can be administered in a patient who has undergone a curative or primary surgery.

A cancer according to the invention is caused by an uncontrolled division of abnormal cells in a part of the body.

The cancer may be a solid cancer or a cancer affecting the blood (i.e., leukemia). Leukemia include for example acute myelogenous leukemia (AML), chronic myelogenous leukemia (CML), acute lymphocytic leukemia (ALL), and chronic lymphocytic leukemia, (including various lymphomas such as mantle cell lymphoma, Hodgkin's lymphoma or non-Hodgkins lymphoma).

Solid cancers typically involve a malignant growth or tumor resulting from an uncontrolled division of cells. Solid cancers notably include cancers affecting one of the organs selected from the group consisting of colon, retina (such as retinoblastoma), rectum, skin (such as melanoma, notably advanced melanoma), endometrium, aerodigestive tract (including laryngeal carcinoma), gallbladder and bile tract, lung (including non-small cell lung carcinoma), uterus, bones (such as Osteosarcoma, Chondrosarcomas, Ewing's sarcoma, Fibrosarcomas, Giant cell tumors, Adamantinomas, and Chordomas), liver, kidney, esophagus, stomach, bladder (including urothelial bladder carcinoma and urinary tract carcinoma), pancreas, cervix, brain (such as Meningiomas, Glioblastomas, Lower-Grade Astrocytomas, Oligodendrocytomas, Pituitary Tumors, Schwannomas, and Metastatic brain cancers), ovary, breast (such as mucinous carcinoma), head and neck region, testis, prostate and the thyroid gland. The term cancer also includes squamous cell carcinoma that may affect the skin, the lungs, the thyroid, the breast, the esophagus or the vagina, as well as fibrosarcoma. In some embodiments melanoma, glioblastomas, aerodigestive tract cancers, breast cancers, lung cancers, urothelial carcinomas, Hodgkin's lymphoma, kidney's cancers, fibrosarcoma, and stomach cancers are preferably targeted by the combination of the present invention.

### Dosage

Preferably the inhibitor of SETDB1 and the immune checkpoint modulator are in an effective dose.

Typically the combined treatment regimen of the invention (i.e., the inhibitor of SETDB1 and the at least one immune checkpoint modulator) is therapeutically effective.

Currently available therapies and their dosages, routes of administration and recommended usage are known in the art and have been described in such literature as the Physician's Desk Reference (60th ed., 2006). Routes of administration include parenterally, intravenously, subcutaneously, intracranially, intrahepatically, intranodally, intraureterally, subureterally, subcutaneously, and intraperitoneally.

Dosage of one or more agents of the invention (e.g., SETDB1 inhibitor and immune checkpoint modulator) can be determined by one of skill in the art and can also be adjusted by the individual physician in the event of any complication.

### Combination Therapies

In a specific embodiment, cycling therapy involves the administration of a first cancer therapeutic for a period of time, followed by the administration of a second cancer therapeutic for a period of time, optionally, followed by the administration of a third cancer therapeutic for a period of time and so forth, and repeating this sequential administration, i.e., the cycle in order to reduce the development of resistance to one of the cancer therapeutics, to avoid or reduce the side effects of one of the cancer therapeutics, and/or to improve the efficacy of the cancer therapeutics.

When two the two combined treatment according to the invention are administered to a patient concurrently, typically in a therapeutically effective regimen the term “concurrently” is not limited to the administration of the cancer therapeutics at exactly the same time, but rather, it is meant that they are administered to a subject in a sequence and within a time interval such that they can act together (e.g., synergistically to provide an increased benefit than if they were administered otherwise). For example, the two therapeutics may be administered at the same time or sequentially in any order at different points in time; however, if not administered at the same time, they should be administered sufficiently close in time so as to provide the desired therapeutic effect, preferably in a synergistic fashion. The combination cancer therapeutics can be administered separately, in any appropriate form and by any suitable route. When the components of the combination cancer therapeutics are not administered in the same pharmaceutical composition, it is understood that they can be administered in any order to a subject in need thereof. For example, a first therapeutically effective regimen can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the second cancer therapeutic as per the invention, to a patient in need thereof.

Preferably the combined administration of an inhibitor of SETDB1 with an immune checkpoint modulator according to the invention leads to a synergistic anti-cancer effect.

### Kit of Parts Preparations

The present application also encompasses preparations containing an inhibitor of SETDB1 as previously described and at least one immune checkpoint modulator as also described above, as a combined preparation for simultaneous, separate or sequential use in cancer treatment. According to such preparations in the form of kit-of-parts” the individual active compounds (i.e., the inhibitor of SETDB1 and the at least one immune checkpoint modulator), represent therapeutic agents and are physically separated, provided that the use of those compounds, either simultaneously, separately or sequentially, produces the new and unexpected joint therapeutic effect as herein described that is not attained by the compounds independently of each other. Indeed as demonstrated by the results below, the claimed combination of active ingredients did not represent a mere aggregate of known agents, but rather a new combination with the surprising, valuable property that the combined anti-tumor effect is much more important that the simple addition of the anti-tumor effects that are observed, when those active ingredients are used separately.

Both active ingredients may be thus formulated into separate compositions or into a unique composition.

The therapeutic agents as per the invention can be suitably formulated and introduced into a subject or the environment of the cell by any means recognized for such delivery.

Such compositions typically include the agent and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration.

### Method of Treatment

The present invention also relates to a method for treating a patient suffering from cancer, wherein said method comprises the combined administration of a SETDB1 inhibitor and at least one immune checkpoint modulator as described previously. Typically, said combined administration is administered according to a therapeutically effective regimen.

The expression of SETDB1 in a patient has been shown to be highly variable, in particular in a patient as defined in the present application (see Cuellar T L et al., JCB 2017, https://doi.org/10.1083/jcb.201612160). The results of the present application now further demonstrate that the activity of an immune checkpoint modulator such as an anti-PD1 or an anti-PDL1 is greatly enhanced in the absence of SETDB1.

Therefore, in one embodiment, the invention also pertains to a method of classifying patient as responsive or not to an immune checkpoint therapy. Typically said method comprises the determination of the level of SETDB1 expression in said patient. The level of SETDB1 expression can be compared to a reference data. Typically if the expression of SETDB1 is lower than said reference data, the patient may be classified as responsive to an immune checkpoint therapy. Alternatively, if the expression of SETDB1 is increased as compared to said reference data, the patient may classified as low responsive to immune checkpoint therapy and could be treated with a combination of an inhibitor of SETDB1 and at least one immune checkpoint modulator as defined in the present application.

Typically the expression of SETDB1 in a patient may be determined from a biological sample from a patient. A biological sample refers to a sample of biological tissue, cells or fluids (such as plasma or blood samples) as classically known in the field.

A reference data may be obtained from the SETDB1 expression determined in a reference sample. Reference sample may be obtained from a subject free of cancer or from the same patient at an earlier time point (for example, before any cancer treatment, or prior the onset of cancer). A reference sample can also typically be obtained by pooling samples from a plurality of subjects to produce a standard over an average population and wherein a standard represents an average level of SETDB1 among a population of individuals. Thus the level of SETDB1 in a standard obtained in such manner is representative of an average level of this marker in a general population or a diseased (typically suffering from a cancer or a specific type of cancer) population.

Detection of SETDB1 can be obtained by any means of detecting expression of a polypeptide or fragment thereof of an mRNA transcript of the polypeptide. Such detection methods are well-known to the one skilled in the art and involve classical protein detection techniques such as immunohistochemistry, Western blot analysis, immunoblotting, ELISA, immunoprecipitation, lateral flow immunoassays, radioimmunoassays and transcript expression level such as measurement of messenger RNA (mRNA) expression through PCR procedures, RT-PCR, Northern blot analysis, RNAse protection assays, etc.

The invention will further be illustrated in view of the following experimental results.

### BRIEF DESCRIPTION OF THE FIGURES

FIG. 1: WT mice were transplanted with WT, Suv39h1^{-/-} or SETDB1^{-/-} B16OVA melanoma cells. When tumors were palpable (2 mm×2 mm), animals were treated with anti-PDL1 therapy and tumor volume measured twice weekly.

FIG. 2: WT C57BL6 mice were transplanted with WT, Suv39h1^{-/-} or SETDB1^{-/-} B16OVA melanoma cells. When tumors were palpable (2 mm×2 mm), animals were treated twice weekly with anti-PD1 antibodies and tumor volume measured twice weekly.

FIG. 3: Loss of Setdb1 in dendritic cells enhances Interferon stimulated gene (ISG) expression and promotes tumor rejection. (a) Expression of ISGs, Ifi204 and Skivl2, in SETDB1^{+/+} (3 histogram bars from the left) and SETDB1^{-/-} (3 histogram bars from the right) bone marrow derived dendritic cells (BMDCs) following LPS treatment for the indicated times. (b) MCA tumor growth in mice in which SETDB1 was conditionally ablated in dendritic cells using the Lox-cre system (CD11c-cre+ SETDB1^{Flox/Flox} (SETDB1^{-/-}) and CD11c-cre-SETDB1^{Flox/Flox} (SETDB1^{+/+}).

FIG. 4: Mice harboring SETDB1^{-/-} dendritic cells are more responsive to anti-PD-1-mediated tumor rejection. SETDB1^{+/+} and SETDB1^{-/-} mice as in FIG. 2 were inoculated with MCA-OVA fibrosarcoma cells and tumor size measured three times per week. PD-1 was administered when tumors became palpable.

FIG. 5: Enhanced tumor rejection in mice with Setdb1^{-/-} dendritic requires CD8+ T cells. MCA-OVA tumors were measured three times per week in SETDB1^{+/+} and SETDB1^{-/-} mice. Anti-CD8 antibody was administered once tumors became palpable.

### EXAMPLES

Mice

A previously described (Collins 2015) mouse strain carrying loxP sites flanking exon 4 of Setdb1 (Setdb1^{tm1a(EUCOMM)Wtsi}) were obtained from EUCOMM and crossed with CD11cre⁺ mice (B6.Cg-Tg(Itgax-cre)1-1Reiz/J; Jackson Laboratory) to generate mice with DC-specific deletion. Setdb1^{tm1a(EUCOMM)Wtsi} mice were also crossed with mice expressing a tomoxifen-inducible cre (Jax, B6; 129-Gt(ROSA)26Sor^{tm1(cre/ERT)Nat/J}) to provide tissue donors for generation of conditional Setdb1^{-/-} BMDCs. ERT-cre⁺ Suv39h1^{WT/WT} bone marrow served as control. C57Bl/6N mice were originally from Charles Rivers Laboratories.

### Cell Culture and Stimulation

Bone marrow-derived dendritic cells were cultivated in 20 ng/ml GMCSF (Miltenyi) in IMDM (VWR13390) supplemented with 10% fetal bovine serum (Eurobio), Penicillin/Streptomycin, 50 μM β-mercaptoethanol, minimal non-essential amino acids, and 2 mM Glutamax (all from Life Technologies) (1-10 medium). Briefly, fresh bone marrow was collected from two of each—ilium, femur, and tibia—by centrifugation. Five million bone marrow cells were seeded on untreated 10 cm plates (VWR) in 10 mls of I-10 medium. On day 3, an additional 10 mls of 1-10 medium was added, followed by collection and replenishment of 10 mls on day 6. BMDC clusters were harvested on day 8 following a 5 minute incubation in PBS (REF) at 4° C. and then stimulated at 2×10⁶ cells per well of an untreated 6-well plate (Sigma M9062-100EA) in 2 mls of 1-10 medium without GMCSF. For generation of Setdb1^{-/-} BMDCs, Cre-mediated deletion was induced by the addition of 20 nM 4-OH-Tamoxifen on day 3 of culture, that was replenished on day 6 and maintained until collection on day 8. Cell stimulations were performed for the indicated times with LPS (100 ng/ml; Invivogen, tlrl-3pelps).

### MCA101 OVA-Expressing Tumor Assay, Immunotherapy, and IFNγ ELISPOT

A previously validated tumor cell line, MCA101-sOVA¹ (fibrosarcoma secreting soluble OVA), was grown in Roswell Park Memorial Institute supplemented with 10% FBS (Eurobio), 100 μg/ml penicillin/streptomycin, β-mercaptoethanol, 2 mM L-glutamine, and hygromycin (Thermo Fisher, 10687010). Cells were harvested by trypsinization of cultures in log-phase growth and resuspended at 10⁵ cells/100 μl of cold PBS for intradermal injection into the right flank of recipient mice. Tumors were visible within 4-5 days and measured every two days hence until they reached 1000 mm³ (calculated as 0.5*W*W*L, W being the width of tumor, and L the length of the tumor). 100 μg of anti-PD-1 (Bio X Cell, RMP1-14) or anti-CD8 (Bio X Cell, 53-6.72) in PBS was delivered by intraperitoneal injection three times per week until the end of the experiment. Blood was collected from mice at day 13 post-tumor inoculation and subjected to rapid (5 seconds) RBCs lysis in sterile H₂O followed by quenching with 10×PBS for a final 1X concentration. 10⁵ cells were plated per well of a pre-coated ELISPOT plate (Fisher Scientific, MAIPS4510) and incubated overnight with MHC I peptide (SIINFEKL; Invivogen OVA 257-264), MHCII peptide (OVA 323-339), or non-specific antigen HSA (human serum albumin) at 37° C. The following day, plates were rinsed in TBS 0.05% Tween20 and IFNγ ELISPOTs were developed following the manufacturer's protocol (ThermoFisher, KMC4021C). Streptavidin alkaline phosphatase purchased from Invivogen and substrate from Bio-Rad (1706432).

### Production of Lentiviral Particles for CRISP R/Cas9 Mutagenesis

HEK293-T cells were maintained in Dulbecco's Modified Eagle's Medium, supplemented with 10% FBS (Eurobio) and 100 μg/ml penicillin/streptomycin. 8.10⁵ were seeded in 6 well plates and transfected with 1 μg psPax2, 0.4 μg VSV-G packaging vector, and 1.6 μg of sgRNA cloned into pCRISP-puro-v2 vectors. Medium exchange was performed 14h post-transfection. Viral supernatants were collected 36h later, filtered and used immediately for transduction of B16-OVA cells.

Sequences for sgRNA used were:

| | F 5′ | F 3′ |
|---|---|---|
| Suv39h | CACCGCCACCTGGGGCGGATCAC | AAACCGGTGATCCGCCCCAGG |
| 1 | CG | TGGC |
| Setdb1 | CACCGCCATAGCTTCACGAAGCT | AAACACAGCTTCGTGAAGCTA |
| | GT3′ | TGGC |
| Sting | CACCGAGCGGTGACCTCTGGGCC | AAACACGGCCCAGAGGTCACC |
| | GT | GCTC |

### Generation of Suv39h1 and Setdb1-Deficient B16OVA Tumor Cells

B16-F10 OVA-expressing melanoma cells were maintained in Roswell Park Memorial Institute (RPMI) supplemented with 10% FBS, 100 μg/ml penicillin/streptomycin and Glutamax®. 2.5.10⁵ were seeded in 6 well plates. 24h after seeding, medium was replaced with 2 ml freshly prepared viral supernatants and plates were spun for 30 min, 2500 rpm in a centrifuge pre-warmed to 30° C. Medium was replaced 24h post-transduction, and puromycin (2 μg/ml, invivogen) added to the cells 48h post-transduction.

Cells were selected with puromycin for two weeks, after which protein expression was checked by western blot (Suv39h1 antibody, Cell Signalling Technology, Setdb1 antibody from Abcam).

For tumor experiments, 2.5×10⁵ tumor cells of the appropriate genotype were injected subcutaneously to C57BL6/J recipients (females aged 6-8 weeks). When tumors were palpable (usually 5 days post-injection), animals were treated twice weekly with 200 μg anti-PDL1 (Bio X Cell, 10F9G2) or an anti-PD1 (PD-1 (Bio X Cell, RMP1-14) 150 μg). Tumors were measured twice weekly using an electronic caliper, and animals were sacrificed when tumors reached 1000 mm³ volume (calculated as 0.5*W*W*L, W being the width of tumor, and L the length of the tumor).

### REFERENCE

1 Zeelenberg, I. S. et al. Targeting tumor antigens to secreted membrane vesicles in vivo induces efficient antitumor immune responses. Cancer research 68, 1228-1235, doi:10.1158/0008-5472.CAN-07-3163 (2008).

### Results

SETDB1^{-/-} B16OVA Cells are More Sensitive to Anti-PDL1 Treatment than WT or Suv39h1^{-/-} B16OVA Cells

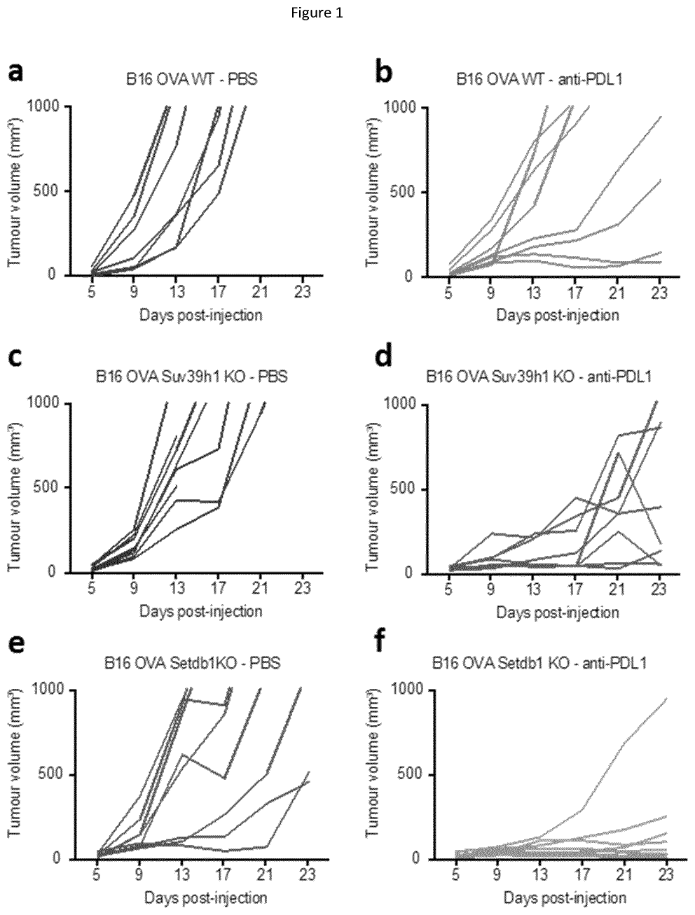

Suv39h1^{-/-} or Setdb1^{-/-} B16 OVA cells, a syngeneic model of murine melanoma grew at a similar rate or slightly faster than WT B16 OVA cells after adoptive transfer in B6 mice (see FIG. 1A vs. 1C).

Treatment with anti-PDL1 was remarkably efficient in inhibiting the growth of Suv39h1^{-/-} or SETDB1^{-/-} B16OVA cells compared to WT controls. Indeed, anti-PD-L1 treatment is inefficient by itself in controlling growth of WT B16OVA cells, and only marginally improves survival.

Treatment with anti-PD-L1 led to a reduction in the growth of Suv39h1^{-/-} B16OVA cells. In sharp contrast, the effect of anti PD-L1 on the growth of SETDB1^{-/-} B16OVA cells was much more drastic, since complete rejection was observed in over 60% of the mice.

The results show that inactivation of SETDB1 in tumor cells increases the efficiency of checkpoint blockade therapy with anti-PDL1 antibodies, and highlight the critical interest of combining Setdb1 inhibition in tumor cells with checkpoint blockade therapy.

### SETDB1^{-/-} B16OVA Cells are Highly Sensitive to Anti-PD1 Treatment as Compared to WT B16OVA Cells

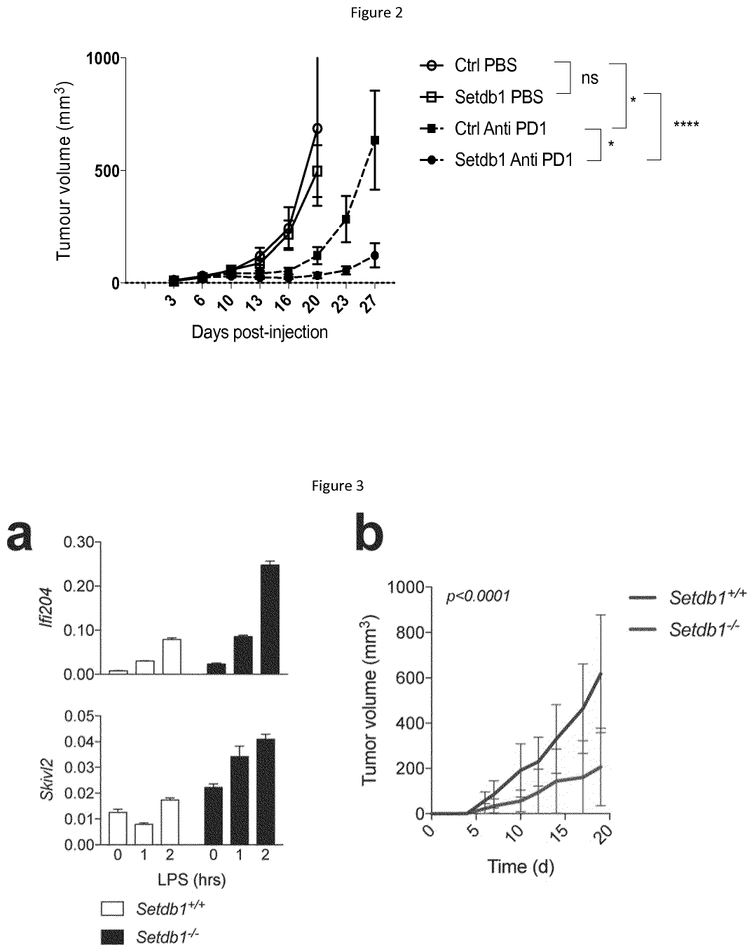

To further explore response of Setdb1-deficient tumors to checkpoint blockade, WT C57BL6 mice were injected with WT or Setdb1-KO B16OVA cells. When tumors were palpable, animals were treated twice weekly with anti-PD1 antibodies. As expected B16OVA cells respond incompletely to treatment with anti-PD1. While Setdb1 deletion in itself does not cause any delay in tumor growth, Setdb1-deficient tumor cells are highly responsive to treatment with anti-PD1 (FIG. 2).

Mice Bearing a Conditional Mutation for Setdb1^{-/-} in Dendritic Cells (DCs) Control Better Tumor Growth and are More Responsive to Anti-Checkpoint Therapy than Control Littermates

Setdb1^{-/-} bone marrow derived dendritic cells (BMDCs) produce more interferon stimulated genes (ISGs) in response to treatment with LPS, indicating a more inflammatory phenotype. In order to test the potential physiological relevance of this phenotype in vivo, we combined CD11c-cre-expressing mice with SETDB1^{Flox/Flox} mice to selectively delete SETDB1 in DCs and inoculated them with MCA-OVA fibrosarcoma cells. Mice that were CD11c-cre-negative served as WT littermate controls. Setdb1^{-/-} mice controlled tumor growth more efficiently than Setdb1^{+/+} mice (FIG. 3). This indicates that the enhanced inflammatory/lsg response in SETDB1^{-/-} myeloid cells promotes better tumor rejection.

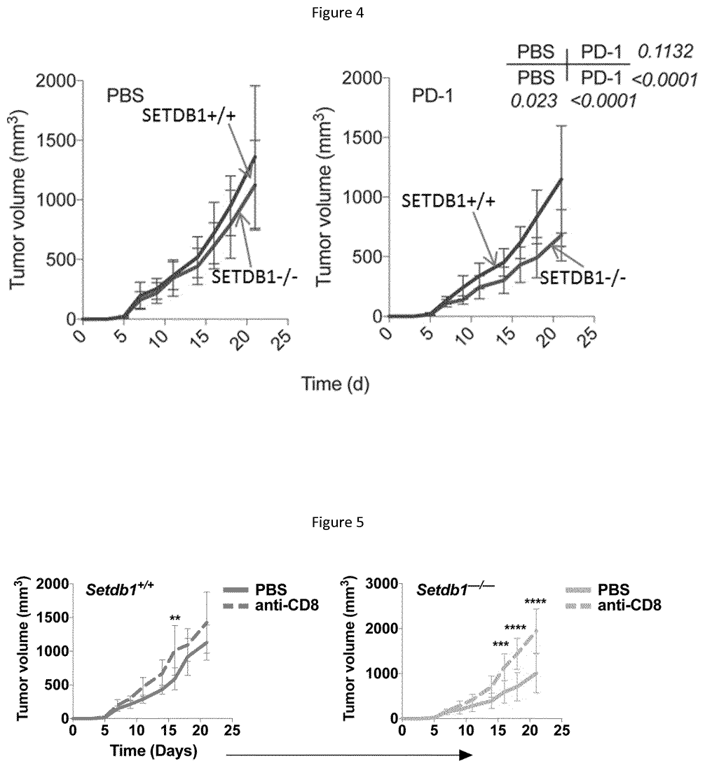

Using the same mouse line with conditional loss of SETDB1 in DCs we performed a similar tumor experiment as in FIG. 3, but administered either anti-PD-1 or PBS as a control (FIG. 4). We observed that Setdb1^{-/-} mice were significantly more responsive to anti-PD-1-mediated tumor rejection, suggesting the potential benefit of combined anti-PD-1 therapy and inhibition of Setdb1 in DCs.

In order to test the requirement for CD8⁺ T cells in enhanced tumor rejection in SETDB1^{-/-} mice, we depleted them by weekly administration of anti-CD8+ antibody. Depletion of CD8⁺ T cells significantly increased the tumor burden in WT and KO animals, requirement for CD8⁺ T cells in control MCA-tumor rejection. Furthermore, these data link the SETDB1^{-/-} phenotype to CD8⁺ T cells (FIG. 5).

1. An inhibitor of H3K9 histone methyl transferase SETDB1 for use in combination with at least one modulator of an immune checkpoint molecule/protein in the treatment of cancer.

2. An inhibitor of H3K9 histone methyl transferase SETDB1 for use according to claim 1, wherein the inhibitor of H3K9 histone methyl transferase SETDB1 is selected from small organic molecules, aptamers, intrabodies, polypeptides or inhibitors of H3K9 histone methyl transferase SETDB1 gene expression.

3. An inhibitor of H3K9 histone methyl transferase SETDB1 for use according to any one of claim 1 or 2, wherein the inhibitor of H3K9 histone methyl transferase SETDB1 is anthramycin.

4. An inhibitor of H3K9 histone methyl transferase SETDB1 for use according to claim 1, wherein the H3K9 histone methyl transferase SETDB1 gene expression is selected from anti-sense oligonucleotide constructs, siRNAs (microRNAs), shRNAs and ribozymes.

5. An inhibitor of H3K9 histone methyl transferase SETDB1 for use in combination with at least one immune checkpoint modulator according to any one of claims 1 to 4, wherein said at least one immune checkpoint is an inhibitory immune checkpoint and/or a stimulatory immune checkpoint.

6. An inhibitor of H3K9 histone methyl transferase SETDB1 for use in combination with at least one immune checkpoint modulator according to claim 5, wherein the inhibitory immune checkpoint is selected from PD-L1/PD1, A2AR, B7-H3, B7-H4, BTLA, CTLA-4, CD277, IDO, KIR, LAG-3, TIM-3 TIGIT, VISTA, CD96, CD112R, CD160, CD244 (or 2B4) DCIR (C-type lectin surface receptor), ILT3, ILT4 (Immunoglobulin-like transcript), CD31 (PECAM-1) (Ig-like R family), CD39, CD73, CD94/NKG2, GP49b (immunoglobulin superfamily), KLRG1, LAIR-1 (Leukocyte-associated immunoglobulin-like receptor 1) CD305, PD-L2 and SIRPα

7. An inhibitor of H3K9 histone methyl transferase SETDB1 for use in combination with at least one immune checkpoint regulator according to claim 5 or 6, wherein the stimulatory immune checkpoint is selected from CD27, CD40, OX40, GITR, ICOS, TNFRSF25, 41BB, HVEM, CD28, TMIGD2, CD226, 2B4 (CD244) and ligand CD48, B7-H6 Brandt (NK ligand), LIGHT (CD258, TNFSF14) and CD28H.

8. An inhibitor of H3K9 histone methyl transferase SETDB1 for use according to any one of claims 1 to 7, wherein said inhibitor is used in combination with at least one inhibitory immune checkpoint modulator and at least one stimulatory immune checkpoint agonist.

9. An inhibitor of H3K9 histone methyl transferase SETDB1 for use in combination with at least one immune checkpoint modulator according to any one of claims 1 to 8, wherein the immune checkpoint modulator is an antibody or a fusion protein.

10. An inhibitor of H3K9 histone methyl transferase SETDB1 for use in combination with at least one immune checkpoint modulator according to any one of claims 1 to 9, wherein the immune checkpoint modulator is an anti-PD-1 or an anti-PD-L1 antibody.

11. A Product containing an inhibitor of H3K9 histone methyl transferase SETDB1 and at least one immune checkpoint modulator as a combined preparation for simultaneous, separate or sequential use in the treatment of cancer.

12. A method for classifying a patient suffering from a cancer as responsive or low responsive to an immune checkpoint therapy, wherein said method comprises the determination of SETDB1 expression in a biological sample from said patient.

13. An inhibitor of H3K9 histone methyl transferase SETDB1 for use according to any one of claims 1-10, a product according to claim 11 or a method according to claim 12 wherein the cancer is selected from melanoma, glioblastomas, aerodigestive tract cancers, breast cancers, lung cancers, urothelial carcinoma, Hodgkin's lymphoma, kidney's cancers, fibrosarcoma, and stomach cancers.
